(19) United States
(12) Patent Application Publication
(10) Pub. No.: US 2022/0409591 A1
(43) Pub. Date: Dec. 29, 2022

(54) Pharmaceutical Formulations for Managing Uric Acid Content in Human Body

(71) Applicant: ZYCIE TECHNOLEGAL LLP, Ahmedabad, Gujarat (IN)

(72) Inventor: Ketana Laljibhai BABARIA, Ahmedabad, Gujarat (IN)

(73) Assignee: ZYCIE TECHNOLEGAL LLP, Ahmedabad, Gujarat (IN)

(21) Appl. No.: 17/775,876

(22) PCT Filed: Nov. 17, 2020
(86) PCT No.: PCT/IB2020/060814

§ 371 Date: May 10, 2022

(30) Foreign Application Priority Data
Nov. 18, 2019 (IN) 201921046867

(51) Int. Cl. A61K 31/426; A61K 9/00; A61K 9/20

(52) U.S. Cl. CPC A61K 31/426; A61K 9/0056; A61K 9/2095; A61K 9/2013; A61K 9/2009; A61K 9/2054; A61K 9/2018

(57) ABSTRACT

The present invention relates to pharmaceutical formulations to treat gout by managing uric acid content in human body by control of making of uric acid in the body and/or removing uric acid from the body. Further, such formulations for treatment of gout are fast available for pharmacological action. Pharmaceutical formulation of the present invention is orally disintegrating solid pharmaceutical dosage form of febuxostat for treating hyperuricemia.

### FIELD OF THE INVENTION

The present invention relates to the field of pharmaceutical formulations for treatment of gout. More particularly, the present invention is related to the field of pharmaceutical formulations for treatment of gout by managing uric acid content in human body by control of making of uric acid in the body and/or removing of uric acid from the body. Furthermore, present invention relates to formulations comprising active pharmaceutical ingredients or its salts or derivatives used to manage uric acid content in human body as mentioned above and overcome prior known problems such as rash, low blood counts, reduced liver function and an increased risk of heart-related death. The formulations of the present invention are fast available for pharmacological action.

### BACKGROUND OF THE INVENTION

Now a days common uneasiness complain comes from adults in which joint pain is more common. It is analyzed that imbalance of diet in human beings and so human body ingredients are also getting imbalanced causes such uneasiness. Many times attack of pain comes that is due to increase of uric acid in blood which deposit around the joints. Uric acid is a product of the metabolic breakdown of purine nucleotides, and it is a normal component of urine. In the purine metabolism uric acid is formed after hypoxanthine and xanthine both metabolized by xanthine oxidoreductase (XOR) effect. Xanthine oxidoreductase is also known as xanthine oxidase (XO). When uric acid is not excreted in urine and too much uric acid remain in blood it will form crystal of uric acid and deposit around the joints. High consumption of purine-rich foods, some types of purine-rich foods, particularly meat and seafood, increases gout risk. Hyperuricemia that is an excess of uric acid in the blood may cause problems such as gout, tophi and kidney stones.

Gout occurs because the body produces too much uric acid and renal clearance capacity decreased, uric acid accumulation in the body, leading to urate crystals deposited in the joints and organs. Some people experience chronic gout, which involves a number attacks occurring over short periods of time. Gout can affect any joint in body. Feet, ankles, knees, and elbows are also common sites of gout. Gout patient have severe pain in joints, joint stiffness, difficulty moving affected joints, redness and swelling and misshapen joints.

If patient has hyperuricemia for several years, uric acid crystals can form clumps called tophi. These hard lumps are found under skin, around joints, and in the curve at the top of ear. Tophi can worsen joint pain and over time damage the joints or compress the nerves. They're often visible to the eye and can become disfiguring.

Uric acid crystals can also cause a buildup of kidney stones. Often, the stones are small and are passed in your urine. Sometimes, they can become too large to pass and block parts of urinary tract. As a result, urinary tract infections are common when kidney stones.

To manage the gout attack and gout-related complications on joins and pain due to this, and to prevent formation of kidney stone and urinary tract infections due to this, medicines are given to reduce making of uric acid in the body or removing uric acid from the body. Approved xanthine oxidase inhibitors are allopurinol and febuxostat which reduce making uric acid and reduce in gout attack and pain due to it. Approved medicines which remove uric acid from body known as uricosurics are probenecid and lesinurad. These medicines play their roles separately for removal or reduction in making so can be given alone or in combination.

Latest disclosed in U.S. Pat. No. 5,614,520 is Febuxostat compound and its use for treatment of gout or hyperuricemia.

Allopurinol (1966) and febuxostat (2009) are xanthine oxidase inhibitor, a drug indicated for use for patients for long term treatment of gout due to high uric acid level. Febuxostat limits the amount of making uric acid in human body and may lower uric acid level in blood and reduce risk of gout. Febuxostat at current highly selective xanthine oxidoreductase (XOR) inhibitors, which act by highly selective to the oxidase and reduce uric acid synthesis as well as reduce uric acid levels so as to effectively treat the disease.

It is disclosed that compared with the traditional treatment of gout drug allopurinol, febuxostat has obvious advantages: (1) allopurinol reduced the XOR only inhibit rather than febuxostat of oxidized and reduced form are XOR significant inhibition, thus reducing the role of uric acid, which is more powerful and lasting; (2) Since allopurinol is a purine analogue, the inevitable result of the purine and other activity related to the impact of pyridine metabolism. So allopurinol treatment should be repeated large doses of the drug to maintain a high level. Febuxostat is a non-purine-selective inhibitor of xanthine oxidase results low dose of drug for the treatment.

In the year 2009, the United States Food and Drugs Administration (USFDA) approved Uloric® as oral tablet 40 mg and 80 mg for the chronic management of hyperuricemia in patients with gout. The active ingredient in ULORIC is 2-[3-cyano-4-(2-methylpropoxy) phenyl]-4-methylthiazole-5-carboxylic acid, with a molecular weight of 316.38 and empirical formula C₁₆H₁₆N₂O₃S. The structural formula of febuxostat is:

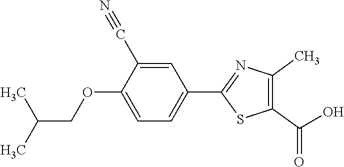 [SMILES: Cc1nc(-c2ccc(OCC(C)C)c(C#N)c2)sc1C(=O)O]

U.S. Pat. No. 6,225,474 claims six crystalline forms of febuxostat viz. crystal A, crystal B, crystal C, crystal D, crystal G and amorphous form of febuxostat. It is described that crystals A, C and G are useful in view of retention of a crystal form in long term storage and among them, crystal A is preferred in view of industrial superiority.

U.S. Pat. No. 7,361,676B2 claims a tablet comprising crystal A, an excipient, and a disintegrating agent. This tablet is prepared by a wet granulating method. However, the tablet is stated as stable and having little variation in the dissolution profiles. In addition to that particular particle size and crystal diameter are the requirement to get desired pharmacokinetic profile.

WO2014009817A1 discloses a solid oral composition comprising micronized febuxostat form-G and one or more pharmaceutically acceptable excipients. In this formulation also polymorphic form G of febuxostat is micronized. Moreover, tablet preparation mentioned in the description is wet granulation.

US20140093563A1 discloses an oral pharmaceutical composition of febuxostat prepared by wet granulation

Indian Application No. 3620/CHE/2010 discloses an amorphous dispersion comprising febuxostat and a dispersing agent and process for preparation of final formulation used is wet granulation. Further the disclosed formulation is not oral dispersible tablet.

This shows that every solid dosage form of febuxostat prepared by wet granulation only. Fast bio availability of febuxostat is not made as fast as it requires to give the effect. As from all these dosage forms febuxostat is absorbed from GI tract and has to go by first pass metabolism.

It is known that bioavailability of febuxostat is more than 80% and has a mean terminal elimination half-life of approximately 5 to 8 hours. It is also known that febuxostat is extensively metabolized by both conjugation via uridine diphosphate glucuronosyltransferase (UGT) enzymes and oxidation via cytochrome P450 (CYP) enzymes. The product label for febuxostat lists hepatic steatosis, hepatitis and hepatomegaly as potential side effects. In, several cases of acute liver failure during febuxostat therapy have been reported to pharmacovigilance databases.

Therefore looking at the existing need, it is desirable to have a dosage form to avoid first pass metabolism of febuxostat and thereby prevent liver damage due to febuxostate. For that reason present invention is now developed orally disintegrating pharmaceutical dosage forms suitable for administering compositions of febuxostat so get fast bio-available of drug to manage uric acid in the body.

### OBJECTS OF THE INVENTION

An object of the present invention is to provide pharmaceutical formulations to manage uric acid in human body by control of making uric acid in the body and/or removing uric acid from the body.

Yet another object of the present invention is to provide pharmaceutical formulations of xanthine oxidase inhibitor and/or uricosurics or its salt or pharmaceutically acceptable derivatives.

Yet another object of the present invention is to provide pharmaceutical formulations of xanthine oxidase inhibitor and/or uricosurics single or in combination.

Yet another object of the present invention is to provide pharmaceutical formulations of xanthine oxidase inhibitor and/or other uricosurics single or in combination and medicines are fast available for pharmacological action.

Yet another object of the present invention is to provide pharmaceutical formulations of xanthine oxidase inhibitor and/or uricosurics which are suitable for all types of patient populations.

Yet another object of the present invention is to provide storage stable formulations of xanthine oxidase inhibitor and/or uricosurics or its salt or pharmaceutically acceptable derivatives.

Yet another object of the present invention is to use the formulations of the present invention in adults for the management of pain due to deposition of uric acid in or around joints.

For management of pain due to deposition of uric acid in or around joints prepare a dosage an object of the present invention is to mange uric acid content in human body by control of making of uric acid in the body and/or removing of uric acid from the body by febuxostat and allopurinol.

To avoid first pass metabolism of febuxostat and febuxostat being poorly water-soluble drug (BCS Class II) need to enhance the solubility of febuxostat and make it fast bio-available to the patient. Accordingly another object of the present invention is to develop orally disintegrating pharmaceutical dosage forms suitable for administering of febuxostat.

According to a yet another object of the present invention is to provide a process for the preparation of orally disintegrating pharmaceutical dosage forms of febuxostat.

Yet another object of the present invention is to use orally disintegrating pharmaceutical dosage forms of febuxostat for treatment of hyperuricemia.

### DETAILED DESCRIPTION OF THE INVENTION

The present invention relates to pharmaceutical formulations for treatment of hyperuricemia wherein treatment of hyperuricemia is by managing uric acid content in human body by control of making of uric acid in the body and/or removing of uric acid from the body. Furthermore, present invention is directed composition comprising active pharmaceutical ingredients or its salts or derivatives for treatment of hyperuricemia mainly known disease due to hyperuricemia is gout which overcome prior known problems such as rash, low blood counts, reduced liver function and an increased risk of heart-related death.

According to one of the further embodiments of the present invention, the oral dosage forms suitable for administering of the present invention comprises xanthine oxidase inhibitor and/or other uricosurics and one or more pharmaceutically acceptable excipients. Xanthine oxidase inhibitor or uricosurics of the present invention comprises febuxostat or allopurinol.

Febuxostat being poorly water-soluble drug (BCS Class II) requirement to enhance the solubility of febuxostat are tried by many prior art including by D. CHRISTOPHER VIMALSON et al., Annamalai University, Chidambaram, Tamil Nadu, India; (Ref: International Journal of Applied Pharmaceutics, Vol 11, Issue 1, 2019) but to make it fast bio-available to the patient and orally disintegrating pharmaceutical dosage forms suitable for administering is not prepared. Accordingly another embodiment of the present invention is to develop orally disintegrating pharmaceutical dosage forms suitable for administering of febuxostat.

As described in the background of the invention it shows that every solid dosage form of febuxostat prepared by wet granulation only. Fast bio availability of febuxostat is not made as fast as it requires to give the effect. As from all these dosage forms febuxostat is absorbed from GI tract and has to go through liver to get available in blood. As described in the foregoing paragraphs, bioavailability of febuxostat is high but extensively metabolized by both conjugation via uridine diphosphate glucuronosyltransferase (UGT) enzymes and oxidation via cytochrome P450 (CYP) enzymes. The product label for febuxostat lists hepatic steatosis, hepatitis and hepatomegaly as potential side effects.

To have a dosage form to avoid first pass metabolism of febuxostat and thereby prevent liver damage due to febuxostat and make fast bioavailability of drug to manage uric acid in the body dosage form of the present invention is designed.

According to one of the further embodiments of the present invention, the orally disintegrating pharmaceutical dosage forms suitable for administering of the present invention comprises febuxostat and at least one pharmaceutically acceptable disintegrant and one or more pharmaceutically acceptable excipients.

In one of the further embodiments of the present invention, orally disintegrating pharmaceutical dosage forms of febuxostat are fast available for pharmacological action may be tablet, film and the like. Furthermore orally disintegrating pharmaceutical dosage forms of febuxostat of the present invention needs to be dosage forms which provide accurate dosing, easy to manufacture, acceptable to patient according to the good mouth feeling even after administration and easy to handle.

In one of the further embodiments of the present invention, orally disintegrating pharmaceutical dosage forms of febuxostat preferred dosage form is tablet. For preparation of orally disintegrating pharmaceutical dosage forms suitable for administering of present invention conventional techniques well known to those skilled in the art can be used such as wet granulation, direct compression, dry compaction (slugging), pellet formation and the like suitable. The term “tablet” refers herein is uncoated compressed pharmaceutical dosage forms of all shapes and sizes. The compressed dosage forms of the present invention can be prepared by any method of compression known by those skilled in the art of producing compressed dosage forms. The compressed dosage forms can also have a different shape than prior known.

In orally disintegrating tablet disintegrating time of tablet is one of the most essential attributes as a proof. The time for disintegration of orally disintegrating tablets is generally considered to be less than one minute. European Pharmacopoeia has used the term orodispersible tablets for orally disintegrating tablets. Other terms for orodispersible tablets are orally disintegrating tablets, mouth-dissolving tablets, rapid-dissolving tablets, fast-disintegrating tablets, fast-dissolving tablets. This may be defined as uncoated tablets intended to be placed in the mouth where they disperse readily within 3 min before swallowing as per European Pharmacopoeia and in-vitro disintegration time is approximately 30 seconds or less as per United States Pharmacopeia (USP). In some embodiments, the dosage forms of the present invention disintegrate in about 60 seconds or less, about 45 seconds or less, about 30 seconds or less. In some embodiments the dosage forms of the present invention disintegrate in about 80 to 90 second, about 110 to 150 seconds, or if more than 150 seconds but not more than 180 seconds.

The technology used in present invention of orally disintegrating tablet of febuxostat can is applicable with any therapeutically effective amount of febuxostat. As used herein, a “therapeutically effective” amount of febuxostat refers to an amount of febuxostat that produces the desired therapeutic response upon oral administration according to a single or multiple dosage regimens for the treatment of hyperuricemia. The precise dosage of febuxostat may vary in relation with the requirement to patient in need of, age, sex and coadministration of other drugs. A therapeutically effective dosage of febuxostat for use with the present invention is about 20 mg, 40 mg, 80 mg, 120 mg per day or as per the requirement to patient in need of it. Orally disintegrating tablet of febuxostat will provide fast availability and rapid action of drug and so will reduce therapeutically effective dose of febuxostat. Febuxostat is present in the dosage forms of the present invention in a concentration of about 20% to about 50% by weight of the dosage form. In some embodiment of the present invention febuxostat is 20 mg and 40 mg.

Total weight of the pharmaceutical dosage forms of the present invention is about 30 mg to about 1000 mg, about 50 mg to about 500 mg, about 50 mg to about 360 mg, about 50 mg to about 240 mg, about 50 mg to about 180 mg, or about 50 mg to about 150 mg. In some embodiments, the pharmaceutical dosage forms of the present invention total weigh is about 60 mg, 80 mg, 110 mg, 120 or about 150 mg. Total weight of the pharmaceutical dosage forms differs in a proportional manner of febuxostat and excipients.

In one of the further embodiments of the present invention, orally disintegrating pharmaceutical dosage forms suitable for administering the pharmaceutically acceptable disintegrant to facilitate the dosage disintegration in the buccal cavity selected from but are not limited to the group comprising colloidal silicon dioxide (AEROSIL®), croscarmellose sodium, crospovidone, sodium starch glycolate (available as PRIMOJEL®, EXPLOTAB®), copolymers of methacrylic acid and divinylbenzene, polacrilin potassium, sulfonated copolymers of styrene and divinylbenzene and combination thereof. Disintegrant is primary excipient in the dosage forms of the present invention is present in a concentration of about 15% to about 60% by weight of the dosage form.

In one of the further embodiments of the present invention, orally disintegrating pharmaceutical dosage forms suitable for administering the pharmaceutically acceptable excipients selected from but are not limited to the group comprising of solubalizer, effervescent agent, diluent, glident, lubricants, pore forming agent, sweetener, flavorant and combination thereof.

Solubilizer of the present invention may be selected from but not limited to the group comprising, sodium lauryl sulfate, polyvinylpyrrolidone, polyethylene glycol, poloxamer, cyclodextrin, Tween 20, Tween 60, Tween 80, polyoxyethylene, polyethylene. Solubalizer is present in the dosage forms of the present invention in a concentration of about 5% to about 15% by weight of the dosage form.

Effervescent agent of the present invention may be selected from but are not limited to the group comprising citric acid, tartaric acid, malic acid, fumaric acid, adipicacid, sodium bicarbonate, sodium carbonate, sodium sesquicarbonate, potassium bicarbonate, potassium carbona. Effervescent agent is present in the dosage forms of the present invention in a concentration of about 0% to about 15% by weight of the dosage form.

Diluent of the present invention may be selected from but not limited to the group comprising, microcrystalline cellulose, mannitol, lactose, dextrin, glucose, sucrose, sorbitol, silicates, calcium and magnesium salts, sodium or potassium chloride. Diluent is present in the dosage forms of the present invention in a concentration of about 2% to about 10% by weight of the dosage form.

Sweetener for use with the present invention to add a sweet taste and are soluble in water of the present invention may be selected from but are not limited to the group comprising acesulfame potassium, aspartame, confectioner's sugar, dextrates, dextrose, fructose, mannitol, saccharin, sorbitol, sucralose, sucrose, xylitol and combinations thereof. Sweetener is present in the dosage forms of the present invention in a concentration of about 2% to about 10% by weight of the dosage form.

Glident as its function to improve flow of the mixture used in the present invention may be selected from but are not limited to the group comprising talc, corn starch. Glident is present in the dosage forms of the present invention in a concentration of about 2% to about 10% by weight of the dosage form.

Lubricants suitable for use with the present invention include, but are not limited to, magnesium stearate, calcium stearate, zinc stearate, sodium stearate, sodium benzoate, stearic acid, aluminum stearate, leucine, glyceryl behenate, sodium stearyl fumarate (e.g., PRUV®, Sohne GmbH & Co., Rosenberg, Germany), hydrogenated vegetable oil, and combinations thereof. Lubricant is present in the dosage forms of the present invention in a concentration of about 0.1% to about 10% by weight of the dosage form.

Optionally in the pharmaceutical dosage forms of the present invention comprise a flavorant can be of a natural or artificial flavoring to improve the taste present invention in a concentration of about 0% to about 10% by weight of the dosage form. Flavorants suitable for use with the present invention include, but are not limited to strawberry, cherry, fruit, almond, citrus, vanilla, coconut, chocolate, camphor, menthol, and combinations thereof.

The pharmaceutical dosage forms of the present invention are substantially free of taste-masking polymers and free of coatings around granules. All these simplifies the manufacturing process, and thus the present invention is also directed to a simple process of preparing febuxostat orally disintegrating dosage forms by a process of dry mixing followed by direct compression.

Methods of Treatment

The present invention is also directed to a method of treating hyperuricemia, orally disintegrating solid pharmaceutical dosage forms to a subject in need thereof including coadministration with other drugs.

Processes to Prepare the Dosage Forms

The present invention is directed to a process of preparing orally disintegrating solid pharmaceutical dosage forms, the process comprising:
(i) Febuxostat and excipients individually passed or premix and mixture is passed through a sieve as per particle size required; here used #22
(ii) Blend well to get uniform mixing.
(iii) Compress the blend into tablet with punch using tablet compression machine; here used 6 mm punch.
(iv) Prepared tablet will be evaluated for different parameter

As used herein, “uniform mixing” refers to the mixtures, compositions, or dosage forms of the present invention having a substantially uniform distribution of ingredients throughout

As used herein, “composition”, ‘blend’ and “mixture” are used interchangeably and refer to a combination of two or more substances.

### Examples

The orally disintegrating dosage forms of the present invention are explained in more detail with reference to the following examples. These examples are provided by way of illustration only and should not be construed as to limit the scope or spirit of the claims in any manner.

TABLE 1
Compositions of orally disintegrating tablet

| | | Weight of Ingredients | | | |
|---|---|---|---|---|---|
| | Role of | Example | Example | Example | Example |
| Ingredients | ingredient | 1 | 2 | 3 | 4 |
| Febuxostat | Active | 40 mg | 40 mg | 40 mg | 40 mg |
| | Ingredient | | | | |
| Sodium Lauryl | Solubilizer | 10 mg | 10 mg | 15 mg | 15 mg |
| Sulfate | | | | | |
| Citric Acid | Effervescent | 30 mg | 15 mg | 20 mg | 30 mg |
| | agent | | | | |
| Microcrystalline | Diluent | 10 mg | 10 mg | 10 mg | 20 mg |
| cellulose | | | | | |
| Colloidal silicon | Disintegrant | 15 mg | 10 mg | 20 mg | 20 mg |
| dioxide | | | | | |
| (AEROSIL ®) | | | | | |
| Croscarmellose | Disintegrant | 15 mg | 10 mg | 20 mg | 20 mg |
| sodium | | | | | |
| Talc | Glident | 5 mg | 5 mg | 5 mg | 5 mg |
| Mannitol | Sweetener | 25 mg | 0 | 10 mg | 0 |

| | | | | |
|---|---|---|---|---|
| Total | 150 mg | 100 mg | 150 mg | 150 mg |
| D.T(second) | >120 | >120 | 30-40 | 60-110 |

*D.T = Disintegration Time

TABLE 2
Composition of orally disintegrating tablet

| | | Weight of Ingredients | | |
|---|---|---|---|---|
| | | | | Example |
| | Role of | Example | Example | 5(C) |
| Ingredient | ingredient | 5(A) | 5(B) | (Placebo) |
| Febuxostat | Active | 40 mg | 40 mg | 0 |
| | Ingredient | | | |
| Poloxamer | Solubilizer | 0 | 0 | 0 |
| (Pluronic F68) | | | | |
| Sodium Lauryl | Solubilizer | 15 mg | 15 mg | 15 mg |
| Sulfate | | | | |
| Citric Acid | Effervescent | 20 mg | 20 mg | 20 mg |
| | agent | | | |
| Sodium | Effervescent | 10 mg | 10 mg | 10 mg |
| Bicarbonate | agent | | | |
| Colloidal | Disintegrant | 20 mg | 20 mg | 20 mg |
| silicon dioxide | | | | |
| (AEROSIL ®) | | | | |
| Croscarmellose | Disintegrant | 20 mg | 20 mg | 20 mg |
| sodium | | | | |
| Microcrystalline | Diluent | 10 mg | 10 mg | 10 mg |
| cellulose | | | | |
| Talc | Glident | 5 mg | 5 mg | 5 mg |
| Sodium | Lubricant | 0 | 0 | 10 mg |
| Benzoate | | | | |
| Mannitol | Sweetener | 10 mg | 10 mg | 20 mg |
| Menthol | Flavorant | 0 | 0 | 10 mg |
| Camphor | Flavorant | 0 | 0 | 10 mg |

| | | | |
|---|---|---|---|
| Total | 150 mg | 150 mg | 150 mg |
| D.T(second) | 35-40 | 35-40 | 35-40 |

In above table duplication of example 5 is carried out for confirmation of the composition along with placebo, this shows disintegration time 35-40 seconds. Further gone to optimize a out of more compositions in Table 3 to get disintegration time less than 30 seconds. Targeting the same example 11 is carried out along with placebo and achieved disintegration time 20-25 seconds.

TABLE 3
Composition of orally disintegrating tablet

| | | Weight of Ingredients | | | |
|---|---|---|---|---|---|
| | Role of | Example | Example | Example 8 | Example |
| Ingredient | ingredient | 6 | 7 | (Placebo) | 8 |
| Febuxostat | Active | 40 mg | 40 mg | 0 | 40 mg |
| | Ingredient | | | | |
| Sodium Lauryl | Solubilizer | 10 mg | 10 mg | 10 mg | 10 mg |
| Sulfate | | | | | |
| Citric Acid | Effervescent | 15 mg | 15 mg | 10 mg | 10 mg |
| | agent | | | | |
| Sodium | Effervescent | 5 mg | 5 mg | 5 mg | 5 mg |
| Bicarbonate | agent | | | | |
| Microcrystalline | Diluent | 5 mg | 15 mg | 5 mg | 5 mg |
| cellulose | | | | | |
| Colloidal silicon | Disintegrant | 15 mg | 15 mg | 15 mg | 15 mg |
| dioxide | | | | | |
| (AEROSIL ®) | | | | | |
| Croscarmellose | Disintegrant | 15 mg | 15 mg | 15 mg | 15 mg |
| sodium | | | | | |
| Talc | Glident | 10 mg | 10 mg | 5 mg | 5 mg |
| Mannitol | Sweetener | 15 mg | 0 | 0 | 0 |
| Sodium Benzoate | Lubricant | 0 | 0 | 0 | 0 |
| Saccharin | Sweetener | 0 | 5 mg | 5 mg | 5 mg |
| Menthol | Flavorant | 10 mg | 10 mg | 5 mg | 0 |
| Camphor | Flavorant | 10 mg | 10 mg | 5 mg | 0 |

| | | | | |
|---|---|---|---|---|
| Total | 150 mg | 150 mg | 80 mg | 110 mg |
| D.T (second) | 35-40 | 30-40 | 20-25 | 20-25 |

As per WHO's Revision of Monograph on Tablets, dispersible tablets shall disintegrate within 3 minutes.

Results of In vitro drug release study of Example 8 using USP II apparatus. In vitro drug release study revealed that % CDR was 50.9511±0.036 at 5 min time interval and 100±0.042 at 10 min interval.

TABLE 5
Drug release report

| Time | % CDR |
|---|---|
| (min) | (mean ± SD) |
| 0 | 0 |
| 5 | 50.9511 ± 0.036 |
| 10 | 100 ± 0.042 |

As is seen above, formulation of the present invention provides pharmaceutically acceptable dissolution profile.

Based on the above study to provide reduced dose of febuxostat a batch is designed as Example 9.

TABLE 6
Example 9

| | Role of | Weight of |
|---|---|---|
| Ingredients | ingredient | Ingredients |
| Febuxostat | Active | 20 mg |
| | Ingredient | |
| Sodium Lauryl | Solubilizer | 5 mg |
| Sulfate | | |
| Citric Acid | Effervescent | 5 mg |
| | agent | |
| Sodium | Effervescent | 2.5 mg |
| Bicarbonate | agent | |
| Microcrystalline | Diluent | 2.5 mg |
| cellulose | | |
| Colloidal silicon | Disintegrant | 7.5 mg |
| dioxide | | |
| (AEROSIL ®) | | |
| Croscarmellose | Disintegrant | 7.5 mg |
| sodium | | |
| Talc | Glident | 2.5 mg |
| Mannitol | Sweetener | 0 |
| Sodium | Lubricant | 0 |
| Benzoate | | |
| Saccharin | Sweetener | 2.5 mg |
| Menthol | Flavorant | 2.5 mg |
| Camphor | Flavorant | 2.5 mg |
| Total | | 60 mg |

TABLE 7
Stability study of the same is done. Batch: Reduce Dose tablet
Tablet weight 60 mg

| | | | |
|---|---|---|---|
| Diameter | Thickness | Hardness | |
| (mm) | (mm) | (kg/cm²) | Disintegration |
| Mean ± SD | Mean ± SD | Mean ± SD | Time (sec) |

0 Day Sample Testing

| | | | |
|---|---|---|---|
| 7.0 ± 0.00 | 2.0 ± 0.00 | 2.13 ± 0.30 | 31.19 ± 1.14 |

One month

| | | | |
|---|---|---|---|
| 7.0 ± 0.00 | 2.0 ± 0.00 | 2.26 ± 0.21 | 31.86 ± 1.48 |

Those who are skilled in the art can understand that some variations in the above described processes can be adopted when one or more other pharmaceutically acceptable excipients are used. A skilled person can change and/or omit sequences of the steps of the described process for the purposes of suitability and convenience where one or more pharmaceutically acceptable excipients may or may not be used without affecting and diminishing the quality and characteristics of the resulting product. Such variations/changes/omissions/additions are well within the scope of the present invention.

1-12. (canceled)

13. An orally disintegrating solid pharmaceutical composition for treating gout, said orally disintegrating solid pharmaceutical composition comprising febuxostat, at least one disintegrant, wherein said orally disintegrating solid pharmaceutical composition disintegrates in about 180 seconds or less.

14. The orally disintegrating solid pharmaceutical composition of claim 13 comprising a pharmaceutically acceptable excipient.

15. The orally disintegrating solid pharmaceutical composition of claim 14, wherein said pharmaceutically acceptable excipient comprises a solubilizer, an effervescent agent, a diluent, a glident, a lubricant, a pore forming agent, a sweetener, a flavorant, or a combination thereof.

16. The orally disintegrating solid pharmaceutical composition of claim 15, wherein said pharmaceutically acceptable excipient comprises said solubilizer in a concentration ranging from about 5% by weight to 15% by weight.

17. The orally disintegrating solid pharmaceutical composition of claim 15, wherein said solubilizer is selected from the group consisting of sodium lauryl sulfate, polyvinylpyrrolidone, polyethylene glycol, poloxamer, cyclodextrin, Tween 20, Tween 60, Tween 80, polyoxyethylene, polyethylene, and combination thereof.

18. The orally disintegrating solid pharmaceutical composition of claim 13, wherein said febuxostat is present in a concentration ranging from about 20% by weight to about 50% by weight.

19. The orally disintegrating solid pharmaceutical composition of claim 13, wherein said disintegrant is present in a concentration of 15% to 60% by weight.

20. The orally disintegrating solid pharmaceutical composition of claim 13, wherein said disintegrant is selected from the group consisting of colloidal silicon dioxide, croscarmellose sodium, crospovidone, sodium starch glycolate, copolymers of methacrylic acid and divinylbenzene, polacrilin potassium, a sulfonated copolymer of styrene, divinylbenzene, and combination thereof.

21. A process for producing an orally disintegrating solid pharmaceutical dosage composition that disintegrates orally in about 180 seconds or less, said process comprising:
admixing febuxostat, at least one disintegrant, and at least one pharmaceutically acceptable excipient to produce a uniformly blended mixture; and
compressing said uniformly blended mixture into a tablet with punch using tablet compression machine to produce said orally disintegrating solid pharmaceutical dosage composition.

22. A method for treating hyperuricemia in a subject, said method comprising administering to the subject in need of such a treatment a therapeutically effective amount of an orally disintegrating solid pharmaceutical composition of claim 1.

23. A method of treating a subject suffering from a clinical condition associated with hyperuricemia, said method comprising administering to the subject in need of such a treatment a therapeutically effective amount of an orally disintegrating solid pharmaceutical composition of claim 1.
